(19) United States
(12) Patent Application Publication
(10) Pub. No.: US 2026/0318183 A1
(43) Pub. Date: Oct. 15, 2026

(54) METHODS AND SYSTEMS FOR DETECTING HYDROCARBON MICROSEEPAGE FROM DEEP GEOLOGICAL FORMATIONS

(71) Applicant: SAUDI ARABIAN OIL COMPANY, Dhahran (SA)

(72) Inventors: Ibrahim Atwah, Dhahran (SA); Vera Solovyeva, Moscow (RU); Maxim Orlov, Moscow (RU)

(73) Assignees: Aramco Innovations LLC, Moscow (RU); SAUDI ARABIAN OIL COMPANY, Dhahran (SA)

(21) Appl. No.: 19/484,825

(22) PCT Filed: May 17, 2023
(86) PCT No.: PCT/RU2023/000145

§ 371 Date: Nov. 14, 2025

(51) Int. Cl. E21B 44/00; E21B 49/06; E21B 49/08; G01N 33/24

(52) U.S. Cl. CPC E21B 44/00; E21B 49/06; E21B 49/088; G01N 33/241; E21B 2200/20

(57) ABSTRACT

A method and system for determining a presence of a hydrocarbon microseepage based on an analysis of a dual soil sample is provided. The method may include collecting a dual soil sample from a borehole using a dual soil sampling system. The dual soil sample may include a soil sample collected with an active soil sampling device and a soil gas sample collected with an umbrella-shaped passive soil gas sampler. The method may further include determining an analysis of the dual soil sample using a dual soil sample processing system and determining a presence of the hydrocarbon microseepage based, at least in part, on the analysis.

### BACKGROUND

In the petroleum industry, hydrocarbons are located in reservoirs far beneath the surface of the Earth. Wells are drilled into these reservoirs to access and produce hydrocarbons. In some cases, hydrocarbons can migrate through faults or fractures upwards towards the surface. Hydrocarbon seepage is a natural phenomenon that occurs as a surface expression of this vertical migration. A hydrocarbon macroseepage occurs when there is a large concentration of hydrocarbons easily detectable at the surface. In comparison, a hydrocarbon microseepage occurs when there are ultra-low trace levels of analytically detectable hydrocarbons in soils, sediments, or waters. Geochemical prospecting for hydrocarbons includes searching for surface or near surface hydrocarbons and their alteration products, which may guide the discovery of oil and gas accumulations from an underlying reservoir.

Conventional geochemical exploration techniques include soil sampling. Soil sampling occurs both directly by collecting a physical sample of the surface or near surface soil, or indirectly by detecting seepage-induced changes to the soil, sediment, microbes or vegetation. Once collected, these soil samples are often delivered to a laboratory for processing and analysis to determine a hydrocarbon microseepage. However, due to the low concentration in a microseepage, detection is more difficult and requires sensitive analytical instruments for accurate measurement. Hydrocarbon microseepage is also often hindered by contamination issues including anthropogenic industrial signal, recent organic matter signal, and soil sediment background signal. Improvements in geochemical prospecting techniques may aid in hydrocarbon exploration as well as soil and groundwater contamination prevention.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments disclosed herein relate to a method for determining a presence of a hydrocarbon microseepage based on an analysis of a dual soil sample. The method may include collecting a dual soil sample from a borehole using a dual soil sampling system. The dual soil sample may include a soil sample collected with an active soil sampling device and a soil gas sample collected with an umbrella-shaped passive soil gas sampler. The method may further include determining an analysis of the dual soil sample using a dual soil sample processing system and determining a presence of the hydrocarbon microseepage based, at least in part, on the analysis.

In general, in one aspect, embodiments relate to a system including a borehole, a dual soil sampling system configured to collect a dual soil sample from borehole that includes an active soil sampling device configured to collect a soil sample in a bottom section of the borehole and an umbrella-shaped passive soil gas sampler configured to collect a soil gas sample in the bottom section of the borehole, and a dual soil sampling processing system. The dual soil sampling processing system may include a fluorescence (XRF) analyzer configured to perform a mineralogical analysis of the soil sample, a liquid extraction device configured to create and extract a liquid soil extract from the soil sample, a thermal desorber configured to extract a desorbed soil gas from the soil gas sample, at least one of a triple-quadruple mass spectrometer or a time-of-flight mass spectrometer configured to determine an analysis of the dual soil sample by determining a first chromatogram from the liquid soil extract and determining a second chromatogram from the desorbed soil gas, and a data processing module configured to determine a presence of a hydrocarbon microseepage based, at least in part, on the analysis.

Other aspects and advantages of the claimed subject matter will be apparent from the following description and the appended claims.

### BRIEF DESCRIPTION OF DRAWINGS

Specific embodiments disclosed herein will now be described in detail with reference to the accompanying figures. Like elements in the various figures are denoted by like reference numerals for consistency. Like elements may not be labeled in all figures for the sake of simplicity.

FIG. 1 depicts a hydrocarbon microseepage site with one or more embodiments.

FIGS. 2A-2B depict a system in accordance with one or more embodiments.

FIG. 3 depicts a dual soil sample processing system in accordance with one or more embodiments.

FIG. 4 shows a flowchart in accordance with one or more embodiments.

FIG. 5 shows a flowchart in accordance with one or more embodiments.

FIGS. 6A-6B show chromatograms in accordance with one or more embodiments.

FIG. 7 depicts a drilling system in accordance with one or more embodiments.

FIG. 8 depicts a neural network in accordance with one or more embodiments.

FIG. 9 depicts a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

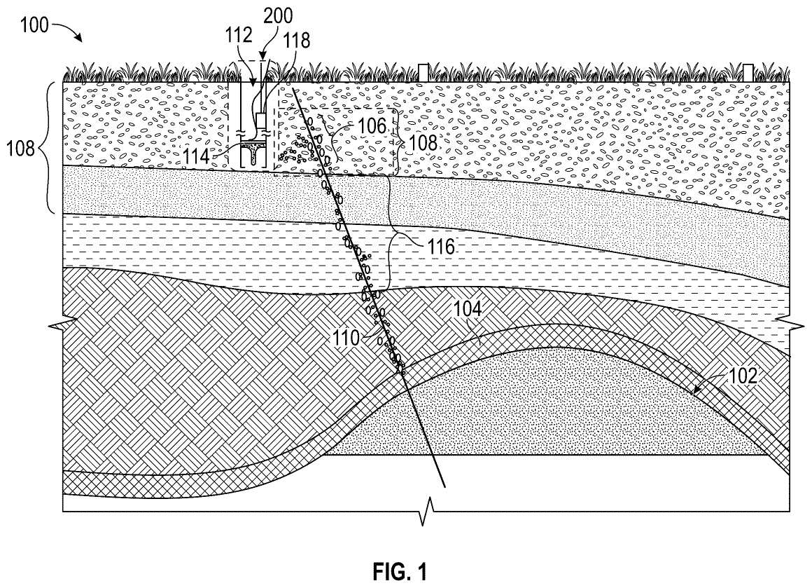

In the following description of FIGS. 1-9, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a passive soil gas sample system” includes reference to one or more of such systems.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

Embodiments disclosed herein include methods and systems for collecting a dual soil sample from a borehole in order to determine the presence of a hydrocarbon microseepage. As used herein, the term “borehole” refers to a shallow borehole typically a few feet to a few tens of feet in depth drilled for the purpose of collecting a dual soil sample. In contrast, the term “wellbore” as used herein refers to a wellbore drilled to produce hydrocarbons, such as oil and gas, drilled to penetrate a hydrocarbon reservoir typically at a depth of a few thousand to a few tens of thousands of feet below the surface of the earth. The methods include taking a dual soil sample made up of a soil sample, possibly using a sidewall auger and a soil gas sample with an umbrella-shaped passive soil gas sampler. The umbrella-shaped passive soil gas sampler includes an outside surface covered in a gas-permeable aquaphobic membrane that allows a passage of the soil gas sample, a gas trapping sorbent material that partially fills the umbrella-shaped passive soil gas sampler and a fluid conduit that connects a top inflatable rubber seal to a fluid pump controlled at a surface location. The umbrella-shaped passive soil gas sampler is deployed at a bottom section of the borehole and the borehole is sealed by inflating the top inflatable rubber seal. The umbrella-shaped passive soil gas sampler remains in the borehole for a sampling period and then is retrieved at the surface for analysis of the contents. The analysis is performed on the dual soil sample by performing a mineralogical analysis on the soil sample, determining a first chromatogram of the soil sample, and determining a second chromatogram of the soil gas sample. The first and second chromatogram are determined by creating a liquid soil extract from the soil sample, extracting a desorbed soil gas from the soil gas sample and using at least one of a triple-quadruple mass spectrometer or a time-of-flight mass spectrometer, determining the first chromatogram from the liquid soil extract and the second chromatogram from the desorbed soil gas.

In some embodiments, the chromatogram of a sample taken from a location affected by a microseepage may differ chromatogram of a sample taken from a location unaffected by a microseepage. To determine a presence of a hydrocarbon microseepage, a training dataset may be obtained. The training dataset may include a collection of known chromatography data from the vicinity of a hydrocarbon producing well and the vicinity of a dry well. In some embodiments, the training dataset may be used to form a first discriminant function to define a characteristic hydrocarbon microseepage signature and a second discriminant function to define a characteristic background signature, using a machine learning (ML) network.

The first chromatogram of the soil sample and a second chromatogram of the soil gas sample may be interpreted using the first and second discriminant function to determine the hydrocarbon microseepage. The determined hydrocarbon microseepage includes information relating to the presence and quantities of determined hydrocarbons. A reservoir model may be generated using a reservoir modeler, based at least in part, on the hydrocarbon microseepage and a reservoir simulation may be performed using a reservoir simulator to determine a drilling target based on the reservoir model. A wellbore path, or trajectory may then be planned, using a wellbore path planning system, to intersect the drilling target and a wellbore is drilled guided by the wellbore path using a drilling system. By combining the analysis performed on the soil sample with the analysis of the soil gas sample, the dual soil sampling technique improves on existing methods to determine a hydrocarbon microseepage by providing a more comprehensive data collection, resulting in a decreased uncertainty of the analysis. Furthermore, including both soil and soil gas samples allows the method to distinguish between current active seepage and historic seepage in the geological past.

FIG. 1 depicts a hydrocarbon microseepage system (100) in accordance with one or more embodiments. The hydrocarbon microseepage system (100) includes an underlying reservoir (102) that contains an accumulation of hydrocarbons including oil and/or natural gas. The reservoir (102) is usually a permeable and porous rock layer, overlain by an impermeable layer or layers of rock, known as a hydrocarbon seal (104) that, combined with an appropriate topographic structure, such as an anticline, traps the accumulation of hydrocarbons within the reservoir (102). A hydrocarbon seepage may occur where the seal (104) fails, such as at a geological fault (110), herein simply a “fault”, or fracture in the seal (104) allows a natural vertical migration of at least a portion of the hydrocarbons from the reservoir (102) to a location (108) at or near the surface of the earth. A hydrocarbon macroseepage refers to visible oil and gas seeps found at a surface location. Macroseeps have large concentrations of hydrocarbons and are usually localized to the termination of faults (110), fractures, and outcropping unconformities or carrier beds. The discovery of new macroseeps is rare, although easily detectable.

A hydrocarbon microseepage (106) in comparison, is both more common and yet more difficult to detect. Depicted in FIG. 1, a hydrocarbon microseepage (106) is characterized by a low concentration of hydrocarbons in soils, sediments, or waters, only analytically detectable by the appropriate sensors, that result from a vertical migration of hydrocarbons from the reservoir (102). The microseepage hydrocarbons may contain volatile or semi-volatile compounds. In these cases of seepage, the hydrocarbons migrate vertically through faults (110) or fractures upwards through overlying rock layers (116) towards a surface or near surface location (108). The fault (110) depicted in FIG. 1, fractures and displaces the hydrocarbon seal (104) allowing for the escape of the hydrocarbons in these cases. Microseepage (106) may also occur as a near-vertical migration from the reservoir (102) due to a buoyancy-driven flow of hydrocarbons. A buoyancy-driven flow of hydrocarbons occurs when hydrocarbons are less dense than the pore fluid located above them. Due to this density difference, the buoyant force exerted on the hydrocarbons causes an upward migration.

Geochemical prospecting for a hydrocarbon microseepage (106) facilitates the discovery of oil and gas accumulations from the underlying reservoir (102) and is usually carried out by soil sampling. Furthermore, detection of a hydrocarbon microseepage (106) may be pertinent in soil or groundwater contamination prevention. In some embodiments, geochemical prospecting may include searching for the presence of surface or near-surface hydrocarbons and their alteration products at levels detectable by the appropriate sensors. Geochemical prospecting includes a wide array of techniques ranging from directly detecting hydrocarbons that have escaped their subsurface accumulations, to identifying secondary responses in the soils, rocks, and microorganisms. Soil sampling may occur both directly by collecting a physical sample of the surface or near surface soil, or indirectly by detecting seepage-induced changes to the soil over time.

FIG. 1 depicts geochemical prospecting on a hydrocarbon microseepage system (100) by soil sampling. A borehole (112), typically a shallow borehole a few meters to a few tens of meters deep, may be drilled in an area of suspected hydrocarbon microseepage (106) in order to detect and quantify the hydrocarbons through various geochemical exploration methods. Geochemical exploration methods may include the deployment of an active soil sampling device (118) down the borehole (112) to a near surface location (108) to collect a soil sample. Active or direct sampling usually includes the use of instruments such as a sidewall auger, a hollow stem auger, or any other instrument or device capable of extracting a soil sample from the borehole (112). Active soil sampling may also be taken during well operations such as a coring operation, a drilling operation or any wireline operation. In comparison, a passive soil sampling device (114) may be deployed inside the borehole (112) to passively or indirectly sample the soil for hydrocarbons. The passive soil sampling device (114) may be disposed in a bottom section of the borehole (112) to remain stationary for a period of time, trapping a soil gas sample. After the period of time has elapsed, often two weeks or more, the passive soil sampling device (114) may be recovered to the surface for analysis. The passive soil sampling device (114) may sample any available hydrocarbon fluids, including hydrocarbon liquids and hydrocarbon gas liquids (HGLs). Once collected, a soil sample may be sent to an offsite location, such as a laboratory, for analysis. In the analysis, a detailed chemical profiling of organic compounds may be performed to determine the presence of a hydrocarbon microseepage (106).

Soil samples are often hindered by contaminants from anthropogenic factors that may include pollutants from agricultural activities, equipment lubricant leakage, chemical waste dumping, mining activities and industrial activities. Due to the low hydrocarbon concentration in a microseepage (106) coupled with frequent contamination from anthropogenic factors, the presence of recent organic matter, and soil sediment background noise, the accurate detection of a hydrocarbon microseepage (106) is challenging. Improvements in geochemical prospecting techniques, such as the embodiments disclosed herein, aids in accurate and robust microseepage detection, that in turn, facilitates hydrocarbon exploration.

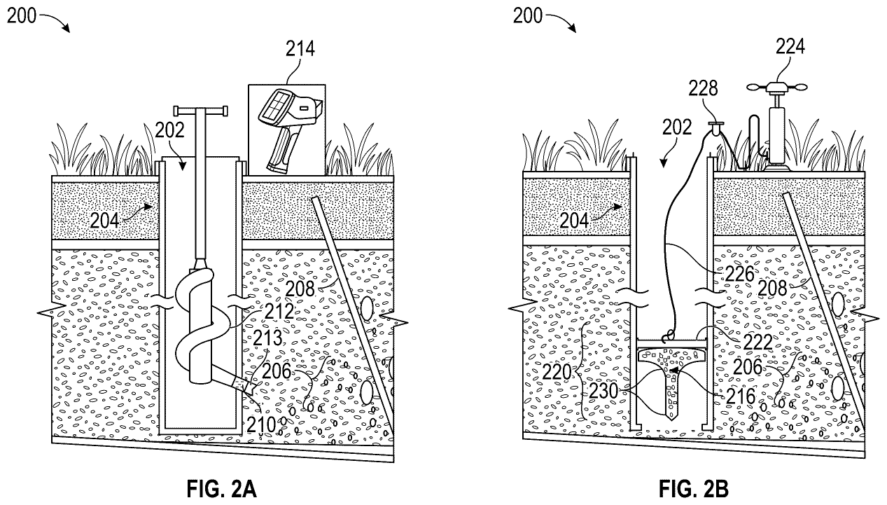

FIGS. 2A and 2B depict a dual soil sampling system (200), in accordance with one or more embodiments, sampling a hydrocarbon microseepage (206) that has migrated from a reservoir, along a path, such as the path created by a fault (208) or fractures. The dual soil sampling system (200) combines two elements, an active soil sampling system, illustrated in FIG. 2A, with a passive soil sampling, illustrated in FIG. 2B. The dual soil sampling system (200) includes a borehole (202) drilled in an area of interest and stabilized with a rigid casing (204). The borehole (202) may be stabilized using a rigid casing fabricated from PVC, stainless steel, carbon steel, or other rigid materials.

In some embodiments, the borehole (202) may be drilled with water-based mud or lubricated with a bio-based oil. Bio-based oils are lubricants derived from plant and vegetable oils that are engineered for hydraulic system operations and are certified for biodegradability and non-toxicity if leaked to the soil or the borehole (202). Importantly, in this context, biobased oils have chemical signatures that differ from the chemical signatures of hydrocarbon derived oils. Due to the low hydrocarbon concentration found in soil samples containing a microseepage (206), the conventional use of petroleum-based lubricants during drilling and casing operations may contaminate the soil samples by creating a false-positive microseepage signature. False-positive results may be detected because petroleum-based lubricants may have a similar chemical composition to a natural microseepage (206). Bio-based oils may also be used to lubricate the drilling equipment involved, such as motors, joints and swivels. Thus, by using water-based muds or bio-based lubricants having a chemical composition easily distinguishable from a natural hydrocarbon microseepage (206), a petroleum contamination is greatly reduced or eliminated.

The depth of the borehole (202) may vary depending on geological settings. Factors controlling the depth of the borehole (202) may include the depth of bedrock, soil conditions including clay content, as well as economic constraints that may limit the ability to drill a deeper borehole. However, shallow boreholes drilled to a depth of 6 feet (~2 meters) or less may be too close to the ground surface and thus, be heavily contaminated by anthropogenic factors present in shallow soils or sediments.

After the borehole (202) is stabilized, the dual soil sample collection may begin by taking a soil sample (210) using, for example, an active soil sampling device (212) illustrated in FIG. 2A. In FIG. 2A the active soil sampling device (212) is shown collecting a soil sample (210) in a bottom section of a borehole (202). In some embodiments, one example of the active soil sampling device (212) may be a sidewall auger. The sidewall auger representing one example of the active soil sampling device (212), is shown in FIG. 2A having a spiral design that when rotated, soil from a bottom section of the borehole wall (202) is scraped off into an auger tip (213). Any other instruments capable of obtaining a soil sample (210) from the bottom section of the borehole (202) may be used, without departing from the scope of the invention, including a hollow stem auger, a core barrel, a diamond coring bit or a rotary drill. In some embodiments, the soil sample (210) may be obtained using sonic drilling technology. Once the soil sample (210) is collected inside the auger tip, the soil sample (210) may be returned to the surface for an analysis. In some embodiments, approximately 100 grams (g) of soil may be collected for the soil sample (210).

A mineralogical analysis may then be performed, in accordance with one or more embodiments. The mineralogical analysis of the soil sample (210) may include using a fluorescence (XRF) analyzer (214). An XRF analyzer (214) irradiates the soil sample (210) with X-rays, causing the elements in the soil sample (210) to fluoresce. The emitted florescent X-rays may travel back to the XRF analyzer's X-ray detector, where it may be measured. In some embodiments, the soil sample (210) may be transported from a soil sampling location to a laboratory to be analyzed by an XRF analyzer (214). In other embodiments, a hand-held XRF analyzer (214) may be used at the sampling location. The analysis determined from the XRF analyzer (214) may be performed as a screening criterion for the soil sample (210), to establish a clay content cut-off. Samples with higher clay content tend to better retain and preserve hydrocarbon microseepage signal. Clay content within the subsurface may vary both vertically and horizontally. In some embodiments, if a low clay content has been determined using the XRF analyzer (214), the borehole (202) may be drilled deeper, and the soil sample (210) may be retaken at progressively deeper sections of the borehole (202) until a suitable soil sample (210) containing a higher clay content is obtained. In other embodiments, a new borehole may be created near the original borehole (202) to search for a suitable soil sample (210).

In some embodiments, the soil sample (210) may be sieved to remove coarse sand and gravel sediments to retrieve fine-grained clay particles. A 325-mesh sieve (44 microns) may be used to sieve the soil sample (210). Once the soil sample (210) has been sieved leaving only fine-grained particles, the sieved soil sample (210) may be stored in a container waiting for further lab analysis. The container may be a glass jar or any other device capable of preserving the integrity of the soil sample (210). In some embodiments, the soil sample (210) may be sent to an offsite location, such as a laboratory to be analyzed immediately after collection, while in other embodiments the soil sample (210) may be preserved in a container to be analyzed at a later time. Once a suitable soil sample (210). has been collected, a passive soil gas sampling may begin.

FIG. 2B illustrates a passive soil gas sampling, using an umbrella-shaped passive soil gas sampler (220), in accordance with one or more embodiments. The umbrella-shaped passive soil gas sampler (220) may be configured to take a soil gas sample (216) from the bottom section of the borehole (202) over a sampling period. The umbrella-shaped passive soil gas sampler (220) may include an outside surface (230) composed, at least in part, of a gas-permeable aquaphobic membrane that selectively allows for a passage of the soil gas sample (216), a gas trapping sorbent material that may partially or wholly fill the umbrella-shaped passive soil gas sampler (220) to trap the soil gas sample (216), and a top inflatable rubber seal (222). Once the umbrella-shaped passive soil gas sampler (220) is at a desired borehole (202) depth, the fluid pump (224) seals the bottom section of the borehole (202) by pumping a fluid through the fluid conduit (226) to inflate the top inflatable rubber seal (222). The top inflatable rubber seal (222) may expand due to this increased fluid pressure and expand to a size that creates a seal for a bottom section of the borehole (202). A venting valve (228) coupled to the fluid conduit (226) may be used to control the pressure from the fluid pump (224). Sealing the bottom section of the borehole (202) may be advantageous for strongly reducing or eliminating contamination from the surface or shallower portions of the borehole (202) during the sampling period. In addition to transporting the fluid supply, the fluid conduit (226) may also be used to lower the umbrella-shaped passive soil gas sampler (220) down the borehole (202) and to retrieve the umbrella-shaped passive soil gas sampler (220) at a surface location after the sampling period. In some embodiments, once the borehole (202) has been sealed at a bottom section by the top inflatable rubber seal (222), the borehole (202) may then also be capped at the surface to further isolate the passive soil gas sampling from atmospheric and surface water contamination.

The design of the umbrella-shaped passive soil gas sampler (220) provides a high surface area that may allow for a faster and more efficient absorption of the soil gas sample (216). Additionally, the semispherical dome shape of the umbrella-shaped passive soil gas sampler (220) may preferentially trap gas-analytes under the dome allowing for the equilibration adsorption of the entire analyte gas mixture. The umbrella-shaped passive soil gas sampler (220) further includes an outside surface (230) covered in a gas-permeable aquaphobic membrane that selectively allows for a passage of the soil gas sample (216) while repelling water vapor. The gas-permeable aquaphobic membrane provides better protection of the sorbent material and blocks any aqueous or moisture build-up from interacting and contaminating the sorbent materials.

The membranes that cover the umbrella-shaped passive soil gas sampler (220) are made from one material or a mixture of materials including engineered hydrophobic fluorinated and non-fluorinated oligomers and polymers. The polymers may include Polyvinylidene difluoride (PVDF), polychlorotrifluoroethylene (PCTFE), polytetrafluoroethylene (PTFE), poly-2,2,4-trifluoro-5-trifluoromethoxy-1,3-dioxole (PTTD), TTD homopolymer, poly (perfluoromethyl vinyl ether) (PMVE), PDD homopolymer, co-polymers of tetrafluoroethylene (TFE) and perfluoropropyl vinyl ether (PFA). The polymers further may include tetrafluoroethylene (TFE), perfluoroprorene (FEP), and co-polymers of perfluoro-2,2-dimethyldioxole (PDD) with TFE. The membranes may also be made from polymer and co-polymers of perfluoro-butenyl vinyl ether (BVE), polyurethanes, silicones, polyesters, polyamides (aromatic polyamide, copolyimide, polyamide hydrazide), polyimides, polyolefins, polyethylene, polypropylene, (PE, PP) et al, polyvinylchlorides (PVC), polybenzimidazole, polysulphones, vinyl polymers, butadiene rubbers, diene rubbers, polyacrylonitrile, polyether sulphones, poly-ether-ether-ketone (PEEK), poly-ether-ketone PEK, polyphenylene sulfides, polystyrenes and combinations of thereof and co-polymers of abovementioned monomers and oligomers.

The sorbents that partially fill the umbrella-shaped passive soil gas sampler (220) to trap the soil gas sample (216) are insoluble materials, or a mixture of materials used to recover fluids through absorption, adsorption or both. Sorbents may be natural organic, natural inorganic, synthetic or a combination of those materials. The sorbents are carefully selected for their advantageous adsorption of hydrocarbons capabilities and may include activated carbons obtained from various raw materials such as peat, brown and mineral coal, anthracite and plant-based material, such as birch and pine wood, walnut, coconut, and pine nut shells. The sorbents may also include activated carbons with added polymer raw materials including synthetic furfural monomer, carbon molecular sieves, polymeric sorbents based on polystyrene and polymethacrylate such as hyper-crosslinked polystyrene and ion-exchange sorbents such as polyvinylidene chloride, zeolites, and other mineral and synthetic adsorbents. The sorbents used are not only suitable for volatile compounds, but also for trapping liquid hydrocarbon and their biodegraded byproduct compounds.

After the umbrella-shaped passive soil gas sampler (220) has remained deployed downhole, collecting the soil gas sample (216), for a sampling period that may typically exceed two weeks, the passive soil gas sampler (220) may be recovered to the surface. Recovery may include removing the borehole cap, and the top inflatable rubber seal (222) may be deflated using the venting valve (228). The umbrella-shaped passive soil gas sampler (220) may then be recovered at the surface for analysis. The recovery may be performed by raising the passive soil gas sampler (220) using the fluid conduit (226) connected to the top inflatable rubber seal (222) from the borehole (202). Once recovered, the umbrella-shaped passive soil gas sampler (220) may be stored and sealed in a gas-impermeable container for further analysis. The umbrella-shaped passive soil gas sampler (220) may be collected in the container to minimize contamination from atmospheric gas and to prevent damage. The soil sample (210) shown in FIG. 2A, may be sent along with the soil gas sample (216) shown in FIG. 2B to a dual soil sample processing system where an analysis may be determined from the dual soil sample. A dual soil sampling system (200) improves on existing methods for geochemical prospecting by providing a more comprehensive data collection, which may be beneficial in increasing the robustness and reducing uncertainty of the analysis performed.

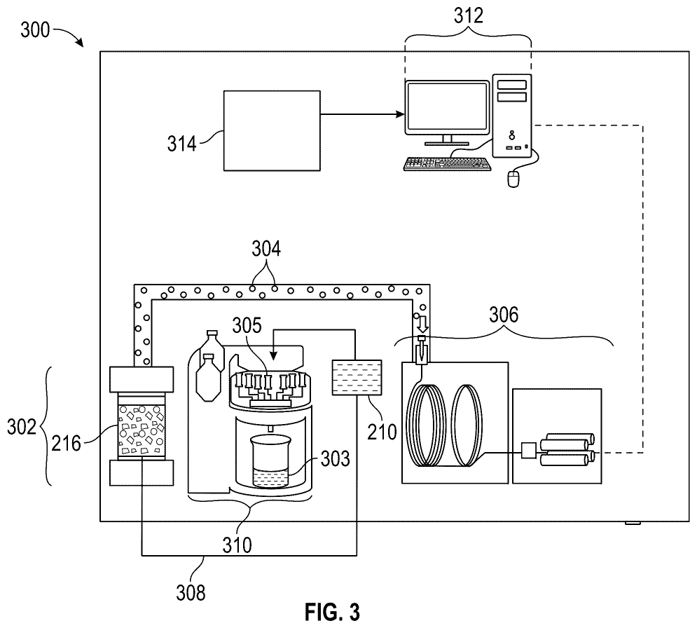

FIG. 3 depicts a dual soil sample processing system (300) in accordance with one or more embodiments. Both the soil sample (210) and the soil gas sample (216) may be sent to the dual soil sample processing system (300) after collection, for a detailed analysis. The dual soil sample (308) may be analyzed using the dual soil sample processing system (300) to determine a presence of known hydrocarbons and other unknown hydrocarbon derivative compounds that may identify a hydrocarbon microseepage. During the first phase of analysis, both sample types may go through an extraction step to isolate the organic compounds including hydrocarbons, from the remainder of the sample.

The dual soil sample processing system (300) may include a thermal desorber (302), configured to extract a desorbed soil gas (304) from the passive soil gas sample (216). The extracted sorbents from the umbrella-shaped passive soil gas sampler (220) may be placed inside the thermal desorber (302), where they are subjected to high temperatures which release soil gas hydrocarbons from the sorbent material holding the soil gas sample (216) into a vapor phase, referred to as desorbed soil gas (304). In some embodiments, the temperature inside the thermal desorber (302) may range from 50-300° Celsius (C). In some embodiments, the thermal desorber (302) may be directly connected to a triple-quadruple mass spectrometer (GC-MS-QQQ) (306) configured to determine the analysis of a dual soil sample (308). In some embodiments, a time-of-flight mass spectrometer (GCxGC-ToFMS) may alternatively be used to perform the analysis.

The soil sample (210) may be prepared for analysis through a solvent extraction process. The soil sample (210) may be placed inside a liquid extraction device (310) configured to create a liquid soil extract (303) in accordance with one or more embodiments. Solvent extraction, or the release of all organic compounds in the soil sediments to the liquid phase, may be achieved by subjecting the soil sample (210) to a high pressure and temperature. The liquid extraction device (310) may be any type of liquid extraction device including a Soxhlet extractor, an accelerated solvent extractor (ASE) and a microwave-assisted extraction (MAE) device. Any device or technique capable of generating a liquid soil extract (303) from the soil sample (210) may be used without departing from the scope of the method.

The soil sample (210) may be placed inside one or more sample vessels (305) included in the liquid extraction device (310) and the sample vessels (305) may be flushed with organic extraction solvents prior to extraction. The recovered liquid after extraction, or the liquid soil extract (303) may contain all organic compounds, including hydrocarbons from a microseepage. The solvents used for flushing may include a combination of dichloromethane, pentane, iso-pentane, hexane, iso-hexane, octane, iso-octane, benzene, toluene, methanol, carbon-disulfide and chloroform. In some embodiments, the liquid soil extract (303) may then be further concentrated by evaporating the extraction solvents down to approximately 1-2 milliliters (ml). The liquid soil extract (303) may be transferred to the GC-MS-QQQ (306) or GCxGC-ToFMS for analysis manually through wet chemistry handling and pipetting. In some embodiments, the transfer may be accomplished through automated robotic arms used for liquid handling.

The liquid soil extract (303) from the soil sample (210) and the desorbed soil gas (304), may each be analyzed via two techniques, including GC-MS-QQQ (306) and/or GCxGC-ToFMS. Gas chromatography is an analytical technique used to separate the chemical components of a sample mixture and then detect targeted compounds and their quantities. Gas chromatography produces a graph called a chromatogram, that shows the patterns and distribution of the identified compounds from the liquid soil extract (303) and the desorbed soil gas (304). Chromatograms present a series of peaks each of which indicates a chemical compound having a specific chemical structure. The GC-MS-QQQ (306) and GCxGC-ToFMS provide sensitive detection of hydrocarbons at ultra-low concentrations (for example, at femtogram-level detection) and have superior selectivity for resolving many compounds in complex matrices such as the liquid soil extract (303) and desorbed soil gas (304). These soil extracts are analyzed for targeted compounds indicative of natural microseepage, which include saturated and aromatic hydrocarbons, polar nitrogen, sulfur, oxygen (NSO) heterocyclic and NSO-derivatives of hydrocarbon compounds, and asphaltenes (and all of their biologically altered counterparts like organic acids and aldehydes). Other known compounds may be measured, which undergo untargeted mass spectra analysis to determine their identity via multiple reaction monitoring and full-scan time of flight mass spectra analysis.

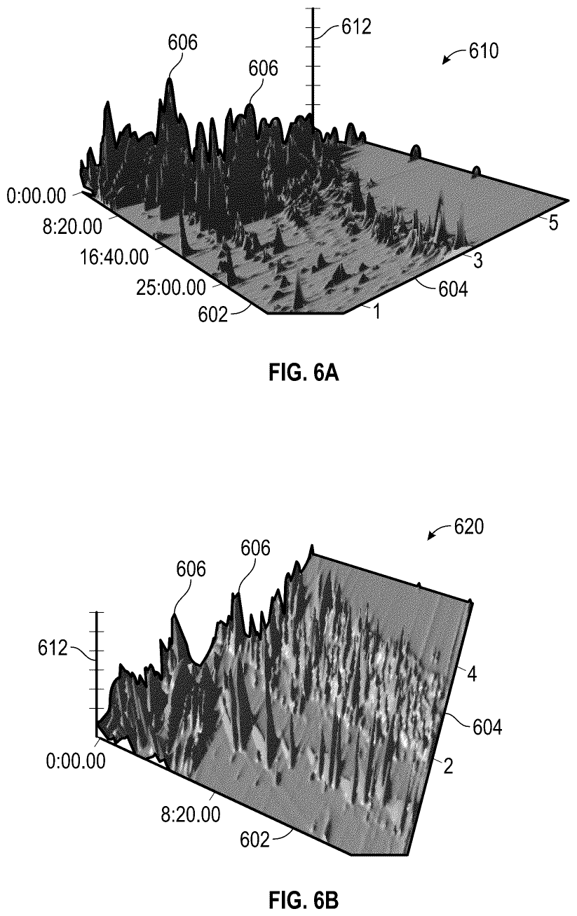

The GC-MS-QQQ (306) or GCxGC-ToFMS are each capable of producing a chromatogram which may be processed and interpreted to determine a hydrocarbon microseepage and either may be used in the method described herein. A first chromatogram is produced from the liquid soil extract (303) originating from the soil sample (210) and a second chromatogram is produced from the desorbed soil gas (304) originating from the soil gas sample (216). The first and second chromatograms may be displayed, processed, and interpreted using a data processing module (312). The chromatograms produced from the dual soil sample processing system (300) are illustrated and discussed further in FIGS. 6A and 6B.

In some embodiments, the first and second chromatograms may be processed by the dual soil sampling processing system (300) using ML techniques to determine a presence of a hydrocarbon microseepage. The data processing module (312) may be configured to accept a training dataset (314) to train a ML network to generate a first discriminant function to define a characteristic hydrocarbon microseepage signature and a second discriminant function to define a characteristic background signature. The first discriminant function may include processed chromatogram data communicating the particular chemical composition expected for a characteristic hydrocarbon microseepage signature in the area of interest. Likewise, the second discriminant function may include processed chromatogram data communicating the particular chemical composition expected for a characteristic background signature in the area of interest. The background signature may include the typical chemical composition expected in the area of interest lacking a hydrocarbon microseepage.

The term processed chromatogram data may describe any data obtained from a chromatogram not in raw form. Therefore, the first and second discriminant functions that include processed chromatogram data may take one of many forms. In some embodiments, the processed chromatogram data may include a list of quantified chemical compounds. In other embodiments, the processed chromatogram data may include a map, a processed chromatogram, or a mathematical function used to fit a peak of a chromatogram. Note that the present disclosure should in no way be limited based on the type of processed chromatogram data that is included in the first and second discriminant functions. The first and second discriminant functions may be used by the data processing module (312) to interpret the first and second chromatograms to determine the presence of a hydrocarbon microseepage.

The training dataset (314) may include data near one or more hydrocarbon producing wells in an area of interest, including a first calibration soil gas sample and a first calibration soil sample. In some embodiments, there may be multiple first calibration samples included in the training dataset (314). The training dataset (314) may further include data near one or more dry wells in an area of interest including a second calibration soil sample and a second calibration soil gas sample. In some embodiments, there may be multiple second calibration samples included in the training dataset (314). In some embodiments, the training dataset may also include reservoir fluid samples from hydrocarbon producing wells and a collection of known chromatography data from the area of interest.

The training dataset (314) including the first and second calibration soil samples and the first and second calibration soil gas samples may be processed by the dual soil sampling processing system (300) to generate calibration chromatograms. These calibration chromatograms may undergo data processing to enhance or isolate the desired chemical components. Data processing may be performed by the data processing module (312) and may include smoothing, baseline corrections, noise reduction, peak integration, and peak alignment among other chromatogram data processing techniques. The data processing may be aided by the reservoir fluid sample in accordance with one or more embodiments. The reservoir fluid sample may be used to determine the distinctive compounds of the actual hydrocarbon fluids in the reservoir of interest and may be used to identify those same compounds on the calibration chromatograms. For example, the reservoir in the area of interest could contain crude or heavy oils or lighter hydrocarbons such as wet gas. These different types of reservoir fluids each have their own distinctive signatures, which may be used as a guide in the data processing to identify a hydrocarbon microseepage signature and a background signature. The calibration chromatograms, which undergo processing aided by reservoir fluid samples and other known chromatography data in the area of interest, are used to train a ML network to generate a first and second discriminant function.

In some embodiments the training dataset (314) may be partitioned into two separate categories based on the sample type prior to generating the calibration chromatograms. Soil samples (210) and soil gas samples (216) exhibit distinct chemical compositions, leading to variations in the chromatograms generated from each. By dividing the training dataset (314) into two separate categories based on their sample type, calibration chromatograms for each sample type may be generated and processed to train the ML network separately. In some embodiments, the training dataset (314) may be separated into a soil gas training dataset that includes the first and second calibration soil gas sample and a soil sample training dataset that includes the first and second calibration soil sample. In some embodiments, the first calibration soil gas sample included in the soil gas training dataset may be processed to create a calibration chromatogram that may indicate a hydrocarbon signature for a soil gas sample in the area of interest. This calibration chromatogram may be processed and then used to train a ML network separately to determine a first discriminant function for a soil gas sample in the area of interest.

The second calibration soil gas sample included in the soil gas training dataset may be processed separately from the first calibration soil gas sample, to create a calibration chromatogram that may indicate a background signal lacking a hydrocarbon signature. This calibration chromatogram may be processed and then used to train a ML network separately to determine a second discriminant function for a soil gas sample in the area of interest.

Likewise, the first calibration soil sample included in the soil sample training dataset may be processed to create a calibration chromatogram that may indicate a hydrocarbon signature for a soil sample in the area of interest. This calibration chromatogram may be processed and then used to train a ML network separately to determine a first discriminant function for a soil sample in the area of interest. The second calibration soil sample included in the soil sample training dataset may be processed separately from the first calibration soil sample to create a calibration chromatogram that may indicate a background signal lacking a hydrocarbon signature. This calibration chromatogram may be processed and then used to train a ML network separately to determine a second discriminant function for a soil sample in the area of interest. In total, there may be four separate types of calibration chromatograms produced from the training dataset (314), each one revealing either the expected hydrocarbon microseepage signature or background signature for both a soil sample and a soil gas sample. In some embodiments, there may be multiple calibration chromatograms created for each type included in the training dataset (314). Training the ML network separately aids in capturing the variations in chromatograms present in the different sample types.

Using the data processing module (312) and the training dataset (314), the ML network may be trained to generate the discriminant functions using a supervised microseepage classification. The discriminant functions may be used to interpret the first and second chromatogram to look for the most abundant compound classes found in the characteristic hydrocarbon microseepage signature and the characteristic background signature to determine a hydrocarbon microseepage. In some embodiments the interpretation may include noise filtering to remove a background signal using the second discriminant function. This noise filtering may subtract the chemical compositions provided in the second discriminant function from the first and second chromatograms. The second discriminant function created from the soil sample training dataset may be used for the first chromatogram and the second discriminant function created from the soil gas training dataset may be used for the second chromatogram. For the noise filtering or subtraction process, the first and second chromatograms may be displayed on the data processing module (312) and the second discriminant functions, may be used to search the first and second chromatograms to identify the background signal for the subtraction.

The first and second discriminant functions that include processed chromatogram data have been described in some embodiments as being a processed chromatogram. In these embodiments, a noise filtering may be performed by overlaying the second discriminant function created from the soil sample training dataset on the first chromatogram using the data processing module (312). The portion of overlap between the first chromatogram and the overlaid second discriminant function may be selected for a subtraction process to remove a background signature from the first chromatogram. Likewise, noise filtering may be performed on the second chromatogram by overlaying the second discriminant function created from the soil gas training dataset on the second chromatogram using the data processing module (312). The portion of overlap between the second chromatogram and the overlaid second discriminant function may be selected for a subtraction process to remove a background signature from the second chromatogram.

In some embodiments, the chemical compositions and concentrations indicated by the second discriminant function may be used to manually select the portions from the first and second chromatograms having a similar composition and concentration for a subtraction. In other embodiments, the ML network may be utilized to identify the background signal using the second discriminant function and remove the background signal automatically. By removing a background signal from the first and second chromatogram, the targeted compounds indicative of a hydrocarbon microseepage may be isolated and more easily identifiable.

With the targeted compounds now isolated, the interpretation of the first and second chromatograms may continue using the first discriminant function. In some embodiments, the first discriminant function may be used to interpret first and second chromatogram to identify similarities in the patterns and distributions of identified compounds. In some embodiments, the peaks observed in the first chromatogram may be identified and then interpreted using the first discriminant function created from the soil sample training dataset. In some embodiments, where the first discriminant function includes a list of quantified chemical compounds, the first chromatogram is interpreted to look for the same quantified chemical compounds to determine a presence of a hydrocarbon microseepage. Positively identifying the same quantified chemical compounds found in the first discriminant function created from the soil sample training dataset in the first chromatogram would indicate a presence of hydrocarbon microseepage in the soil sample. Positively identifying the same quantified chemical compounds in the first discriminant function created from the soil gas training dataset in the second chromatogram would indicate a presence of hydrocarbon microseepage in the soil gas sample.

In other embodiments, where the first discriminant function includes a processed chromatogram, the first discriminant function created from the soil sample training dataset may be overlaid on the first chromatogram using the data processing module (312). An overlap between the first discriminant function and the peaks observed in the first chromatogram would indicate a presence of a hydrocarbon microseepage in the soil sample. Likewise, the first discriminant function created from the soil gas training dataset may be overlaid on the second chromatogram using the data processing module (312). An overlap between the first discriminant function and the second chromatogram would indicate a presence of a hydrocarbon microseepage in the soil gas sample. In some embodiments, the first discriminant functions overlaid on the chromatograms may be interpreted manually to identify the overlap and in other embodiments, the ML network may be utilized to identify the overlap automatically.

In other embodiments the interpretation of the first and second chromatograms may include using the first discriminant function and a classification technique. The classification technique may include multivariate statistical algorithm methods including principal component analysis, hierarchical cluster analysis, canonical variants, neural net classification, and linear discriminant analysis. These classification techniques are pattern recognition ML methods for finding similarities and differences in a dataset. Using any of the forementioned classification machine learning methods, the calibration chromatograms may be used to train the ML network and the trained ML network may be used to identify a characteristic hydrocarbon signature in the first and second chromatograms automatically.

The forementioned classification ML methods are forms of supervised ML and may also include linear regression, logistic regression and decision trees. In these embodiments that use a ML classification technique, the ML network would be trained using the training dataset (314) to accept a first and second chromatogram and automatically identify each pixel's class label using the first and second discriminant functions. The class labels for the first and second chromatogram includes a hydrocarbon microseepage signature or a background signature. In these embodiments, a binary score may be generated for each pixel. For example, a score of 1 may indicate a hydrocarbon microseepage and a score of 0 may indicate a background signal. A binary score metric may be established, using the training dataset (314) and a presence of a hydrocarbon microseepage may be determined based, at least in part, on this metric. In some embodiments, the binary score metric may represent a total number of pixels or a certain percentage of pixels belonging to a hydrocarbon microseepage. If the first or second chromatograms meet or exceed this binary score metric, a presence of a hydrocarbon microseepage may be determined.

In some embodiments, the classification ML methods may be probabilistic classifications. In probabilistic ML classifications, the output of the model is a probability distribution over the possible class labels for each pixel, with each probability representing the likelihood of the pixel belonging to a particular class. In these embodiments, a certainty benchmark may be established that provides the lowest acceptable percentage for a pixel to be confidently identified in a particular class label. For example, a certainty percentage may be established at 80% and each pixel that includes a probability distribution at or above 80% for a certain class label is given a respective binary classification. A binary score metric may then be used to determine the presence of a hydrocarbon microseepage based, at least in part, on the binary score metric.

In other embodiments, a weighted metric may be used to determine the presence of a hydrocarbon microseepage. The soil sample (210) and the soil gas sample (216) may produce chromatograms that are dominated by a background signature, however, still contain true hydrocarbon microseepage signatures. The first and second discriminant functions may be a weighted function that assigns a higher weight to the chemical compositions identified in the first and second chromatograms indicative of a hydrocarbon microseepage. Using a weighted metric, a hydrocarbon microseepage may be identified using a ML classification technique even when the total number of pixels or the total percentage of pixels containing a hydrocarbon microseepage is much less than the total number or percentage of pixels containing a background signature. Using a weighted metric may provide a more accurate assessment of the ML networks performance on imbalanced datasets and help to prevent the ML network from being biased towards the majority class or the background signature.

Only a few supervised classification ML methods have been described to generate the first and second discriminant functions used to evaluate the first and second chromatograms. The discriminant functions may take the form of an explicit relationship, or the weights within a trained ML network. Note that the present disclosure should in no way be limited based on the type or methodology of the discriminant functions described herein.

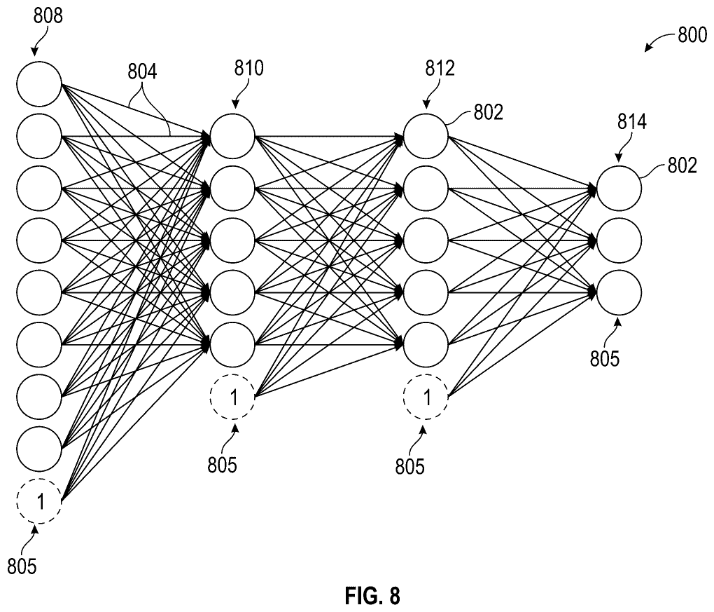

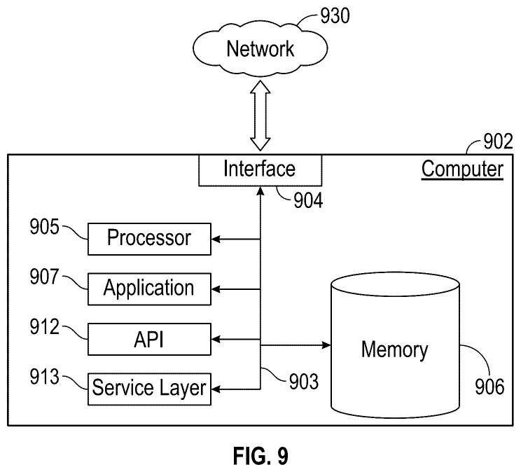

In some embodiments, unsupervised ML techniques, such as clustering ML may also be used. In these embodiments using a clustering ML technique, the training dataset (314) may not be needed. Instead, this unsupervised form of machine learning attempts to identify the targeted compounds by their clustering patterns on the first and second chromatogram. Any ML method may be used to determine the hydrocarbon microseepage signal in accordance with one or more embodiments without deviating from the scope of the method. For example, the machine learning techniques may utilize neural networks, which are depicted and described in further detail in FIG. 8. The data processing module (312) may include a computer system configured to perform these machine learning techniques and is similar to the computer system (902) described below with regard to FIG. 9 and the accompanying description. While the training dataset (314) is not necessary to determine a hydrocarbon microseepage from the first and second chromatogram, incorporating the training dataset (314) in the analysis will increase the confidence in detecting hydrocarbon anomalies that are specific to the area of interest.

In some embodiments, the first and second chromatogram may be interpretated and processed separately using these ML techniques. The first chromatogram may use a ML network that has been trained with calibration chromatograms generated from the soil sample training dataset to determine a hydrocarbon microseepage. Similarity, the second chromatogram may use a ML network that has been trained with the calibration chromatograms generated from the soil gas training dataset to determine a hydrocarbon microseepage. Determining a microseepage includes the identification of a presence of at least one targeted microseepage compound. Quantification of the targeted compounds may be performed and reported in parts per million (ppm).

The quantification of these targeted compounds found from the first and second chromatograms may be compared to one another to determine a current active seepage compared to past historical seepage. This comparison may aid in determining the presence of hydrocarbon accumulations in the subsurface. Furthermore, in some embodiments where no training data is available, the dual soil sampling method may be used to corroborate geologic data in an area of interest to reduce an oil exploration risk. The dual soil sampling method may be used in the cases of no training data, to define locations with surface hydrocarbon anomalies, which may be combined with other geologic data to define a hydrocarbon reservoir.

In some embodiments, the hydrocarbon microseepage determined from the dual soil sampling method may be used by a reservoir modeler, to generate a reservoir model based, at least in part, on the hydrocarbon microseepage determined from the dual soil sample processing system (300). The reservoir modeler may combine the determined hydrocarbon microseepage information, which includes information relating to the presence and quantities of determined hydrocarbons, with other information relating to the reservoir formation obtained from well logs and geological models to create a reservoir model. In some embodiments, a reservoir model may alternatively be created by georeferencing the determined hydrocarbon microseepage to assign a subsurface location of the reservoir from which the hydrocarbon microseepage originated. In these embodiments, two dimensional (2D) maps of the microseepage concentration over the area of interest may be created and overlaid with other geophysical and geological data to infer the seeping reservoir and hydrocarbon accumulations in the subsurface. The reservoir model may include the location of the hydrocarbon reservoir, a location and concentration of the determined microseepage, and a geological map that contains mapped fault structures used to infer a path that hydrocarbon has migrated from the reservoir.

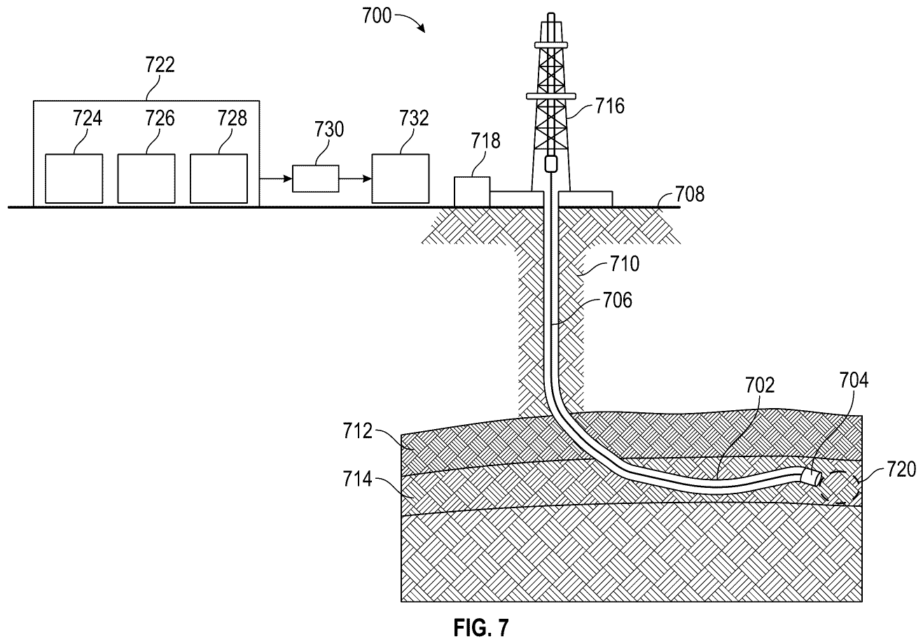

A reservoir simulation may be performed, using a reservoir simulator, to determine a drilling target based on the reservoir model. The reservoir simulation may be performed to predict the behavior of the rocks and fluid under various hydrocarbon recovery scenarios to determine a preferred location to penetrate the hydrocarbon reservoir for economic recovery. A wellbore path may be planned, using a wellbore path planning system, to intersect the drilling target, and a drilling system may be used to drill the wellbore guided by this wellbore path. The reservoir modeler, reservoir simulator, wellbore path planning system, and drilling system are illustrated and discussed further in FIG. 7.

Furthermore, in addition to determining a hydrocarbon accumulation in an area of interest, the dual soil sampling method may also be used for contamination monitoring, geological gas storage monitoring and gas leakage monitoring. By determining the amount of seepage in a soil sample compared to the amount of seepage in a soil gas sample, a leakage of gas can be successfully quantified and used for monitoring purposes.

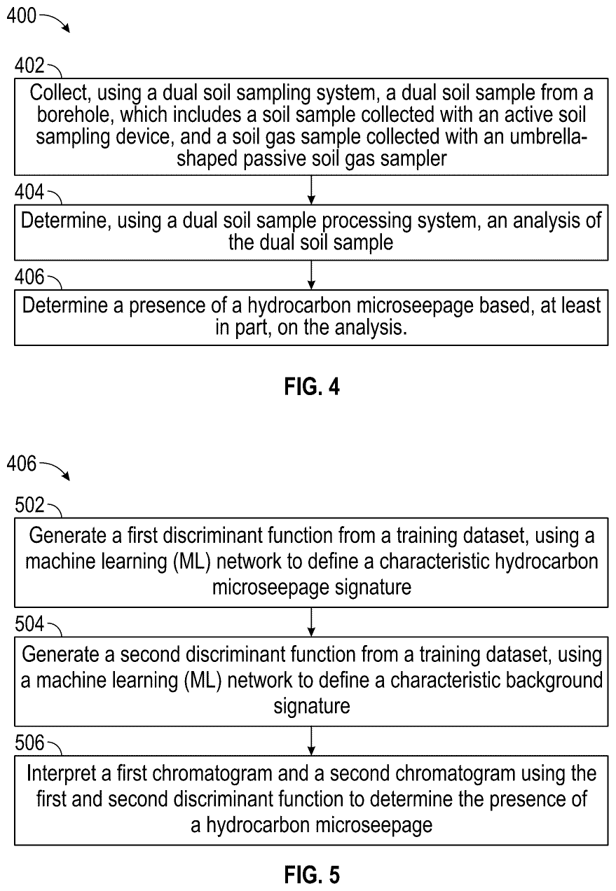

FIG. 4 shows a flowchart (400) in accordance with one or more embodiments. Initially, in Step 402, a dual soil sample may be collected from a borehole using a dual soil sampling system. The dual soil sample may include a soil sample collected with an active soil sampling device, and a soil gas sample collected with an umbrella-shaped passive soil gas sampler. Collecting the dual soil sample may further include stabilizing the borehole using a rigid casing lubricated with a bio-based oil prior to collecting the soil sample. Collecting the soil gas sample with the umbrella-shaped passive soil gas sampler may include deploying the umbrella-shaped passive soil gas sampler in the bottom section of the borehole and sealing the bottom section by inflating a top inflatable rubber seal of the umbrella-shaped passive soil gas sampler. Collecting the soil gas sample may further include taking the soil gas sample over a sampling period, that may exceed two weeks, before retrieving the umbrella-shaped passive soil gas sampler for analysis.

The umbrella-shaped passive soil gas sampler may include an outside surface covered in a gas-permeable aquaphobic membrane that selectively allows for a passage of the soil gas sample, a gas trapping sorbent material that partially fills the umbrella-shaped passive soil gas sampler to trap the soil gas sample, and a fluid conduit connecting the top inflatable rubber seal to a fluid pump controlled at a surface location. The types of membranes and sorbents used are outlined in FIGS. 2A and 2B.

In Step 404, an analysis of the dual soil sample may be determined, using a dual soil sample processing system. Determining the analysis of the dual soil sample may include performing a mineralogical analysis on the soil sample using a fluorescence (XRF) analyzer, determining a first chromatogram of the soil sample, and determining a second chromatogram of the soil gas sample. In some embodiments, the mineralogical analysis may be performed as a preconditioning step, to ensure the soil sample is fit for analysis. The soil sample may then be sieved to remove coarse sand and gravel sediments to retrieve fine-grained clay particles, prior to determining the first chromatogram. Determining the first chromatogram and the second chromatogram may include creating a liquid soil extract from the soil sample and extracting the liquid soil extract using a liquid extraction device, extracting a desorbed soil gas from the soil gas sample using a thermal desorber, and using at least one of a triple-quadruple mass spectrometer or a time-of-flight mass spectrometer to determine the first chromatogram from the liquid soil extract, and determine the second chromatogram from the desorbed soil gas.

In Step 406, a presence of a hydrocarbon microseepage may be determined based, at least in part, on the analysis. Determining the presence of the hydrocarbon microseepage may include, using a data processing module, generating a first discriminant function from a training dataset, using a machine learning (ML) network, to define a characteristic hydrocarbon microseepage signature, generating a second discriminant function from the training dataset, using the ML network, to define a characteristic background signature, and interpreting the first chromatogram and the second chromatogram using the first and second discriminant function to determine the hydrocarbon microseepage. Chromatograms displaying a series of peaks that each represent a chemical compound having a specific chemical structure and are illustrated and discussed further in FIGS. 6A and 6B. Generating the discriminant function from the training dataset and using it to interpret a first and second chromatogram to determine the presence of the hydrocarbon microseepage is discussed further in FIG. 5.

FIG. 5 shows a flowchart (500) in accordance with one or more embodiments. The flowchart (500) describes Step 406 from FIG. 4 in more detail and describes determining a presence of a hydrocarbon microseepage based, at least in part, on the analysis determined from Step 404. In Step 502, a first discriminant function is generated from a training dataset, using a machine learning (ML) network to define a characteristic hydrocarbon microseepage signature and in Step 504 a second discriminant function from a training dataset is generated, using the ML network to define a characteristic background signature. The first and second discriminant functions may include a processed chromatogram data and may take one of many forms including a list of quantified chemical compounds, a map, a processed chromatogram, or a mathematical function used to fit a peak of a chromatogram.

In some embodiments, the first discriminant function may comprise a processed chromatogram communicating the particular chemical composition expected for a characteristic hydrocarbon microseepage signature in the area of interest. Likewise, the second discriminant function may comprise a processed chromatogram communicating the particular chemical composition expected for a characteristic background signature in the area of interest.

The training dataset may include a collection of known chromatography data in an area of interest, a reservoir fluid sample, a first calibration soil gas sample and a first calibration soil sample near a hydrocarbon producing well in the area of interest, and a second calibration soil sample and a second calibration soil gas sample near a dry well in the area of interest. In some embodiments, the training dataset may be separated into a soil gas training dataset that includes the first and second calibration soil gas samples and a soil sample training dataset that includes the first and second calibration soil samples. These training datasets may be developed separately to train the ML network to generate the first and second discriminant functions for both a soil gas sample and a soil sample.

The training dataset may be separated into their sample types and processed by the dual soil sampling processing system to generate calibration chromatograms for each sample type. These calibration chromatograms, which may communicate the chemical compositions of each sample type, may then be used in combination with any other known chromatography data in the area of interest to train a ML network to generate the first and second discriminant functions. The ML network may be trained using these calibration chromatograms determined from the training dataset using a supervised or unsupervised ML technique.

In Step 504, the first chromatogram and a second chromatogram is interpreted using the first and second discriminant function to determine the presence of a hydrocarbon microseepage. The first and second discriminant function may be used to look for the most abundant compound classes found in the characteristic hydrocarbon microseepage signature and the characteristic background signature. Interpreting the first chromatogram and the second chromatogram may include, for both the first chromatogram and the second chromatogram, removing a background signal using the second discriminant function, and using the first discriminant function and a classification technique to identify and quantify at least one targeted microseepage compound. By first removing the background signal from the first and second chromatogram, the targeted compounds indicative of a hydrocarbon microseepage may be isolated and more easily identifiable. Further, the classification technique may be selected from a group consisting of a multivariate statistical algorithm method including, principal component analysis, hierarchical cluster analysis, canonical variants, neural net classification, and linear discriminant analysis. The forementioned classification techniques include a supervised ML technique and may also include linear regression, logistic regression and decision trees. In some embodiments, unsupervised ML techniques, such as clustering ML may also be used.

FIGS. 6A and 6B illustrate 2D representations of 3D chromatograms created from the analysis of a dual soil sample in accordance with one or more embodiments. The chromatograms of 6A and 6B were measured and produced by a GCxGC-ToFMS instrument. Gas chromatography performed by a GCxGC-ToFMS describes a separation technique used to isolate volatile components of a mixture and in this case is used to determine a hydrocarbon microseepage signature in a dual soil sample. Using a GCxGC-ToFMS, samples are vaporized or liquified and injected into a separation column of the GCxGC-ToFMS, which is packed with a finely divided solid or a film of liquid. When the vaporized sample traverses the column, its components may be separated due to differences in their interactions within the column. Upon elution from the column, the separated components may pass over a detector that generates a signal corresponding to the concentration of the compound. The chemical composition may be qualitatively identified based on the delay in the sample passing through the column or the retention time. In some embodiments, the sample may pass through a second column to reveal additional information regarding the sample.

FIG. 6A illustrates a first chromatogram (610), determined from the liquid soil extract of a soil sample and FIG. 6B illustrates a second chromatogram (620), determined from the desorbed soil gas of a soil gas sample. The chromatograms (610,620) were created from a dual soil sample in an area of known hydrocarbon microseepage. The first chromatogram (610) presents the patterns and distribution of chemical compounds that were determined from the soil sample collected near a reservoir. The second chromatogram (620) presents the patterns and distribution of chemical compounds that were absorbed with the prototype umbrella-shaped passive soil gas sampler upon exposure to the reservoir fluid. The chromatograms (610,620) illustrate the determined chemical compounds from a dual soil sample having a retention time through a first GCxGC-ToFMS column illustrated on axis (602). The retention time through the first column may separate chemical components based, at least in part, on boiling point and molecular weight. The chromatograms (610,620) are illustrated with a retention time through a second GCxGC-ToFMS column illustrated on axis (604) which may separate compounds based on their polarity. The relative abundance of each chemical compounds is given by the z axis (612) with the highest concentrations illustrated as a peak, such as one of the peaks (606). The peaks (606) may be interpreted using a training dataset, or other known chromatography data, to determine targeted compounds indicative of a hydrocarbon micro seepage. For example, these chromatograms (610,620) may be interpreted using the first and second discriminate functions to determine a presence of hydrocarbon microseepage using the methods described herein.

FIG. 7 depicts a drilling system (700) in accordance with one or more embodiments. As shown in FIG. 7 a wellbore path (702) may be drilled by a drill bit (704) attached by a drillstring (706) to a drill rig (716) located on the surface of the Earth (708). The well may traverse a plurality of overburden layers (710) and one or more cap-rock layers (712) to a drilling target (720) within a hydrocarbon reservoir (714). The wellbore path (702) may be a curved well path, or a straight well path. All or part of the wellbore path (702) may be vertical, and some well paths may be deviated or have horizontal sections.

Prior to the commencement of drilling, the presence of a hydrocarbon microseepage (724) may be determined from the dual soil sampling method. Further a drilling target (720) may be determined base, at least in part, on the determined microseepage (724). A reservoir model (730) may be generated, using a reservoir modeler (722) based, at least in part, on the hydrocarbon microseepage. In some embodiments, a reservoir modeler (722) comprises functionality for simulating the flow of fluids, including hydrocarbon fluids such as oil and gas, through a formation composed of porous, permeable reservoir rocks. The reservoir modeler (722) may combine information determined from any available well logs (726), the hydrocarbon microseepage (724) determined from the dual soil sampling method and any other geological models (728) available to build models of the reservoir. Well logs (726) may provide depth measurements of a well that describe such reservoir characteristics as formation porosity, formation permeability, resistivity, water saturation, and the like. A geologic model (728) is a spatial representation of the distribution of sediments and rocks (rock types) in the subsurface. The reservoir models (730) may include information regarding total hydrocarbon in place, where the hydrocarbons are located, and how effectively the hydrocarbons can potentially flow. A reservoir simulation may be performed using a reservoir simulator (732) to determine a drilling target (720) based, at least in part, on the reservoir model (730). A reservoir simulation may be used to predict the behavior of rocks and fluid under various hydrocarbon recovery scenarios, allowing reservoir engineers to understand which recovery options offer the most advantageous hydrocarbon recovery plan for a given reservoir (714). A drilling target (720), or a chosen location to penetrate the hydrocarbon reservoir (714), may be determined through reservoir simulation by estimating the fluid flow within the reservoir (714) given various drilling target scenarios.

The reservoir simulator (732) may include hardware and/or software with functionality for performing one or more reservoir simulations regarding determining the drilling target (720) in the reservoir (714). The drilling system (700) may also include a wellbore path planning system (718). A wellbore path (702) may be planned, using a wellbore path planning system, to intersect the drilling target (720). The wellbore plan may include a starting surface location of the wellbore, or a subsurface location within an existing wellbore, from which the wellbore may be drilled. Further, the wellbore plan may include a drilling target (720) and a planned wellbore path from the starting location to the drilling target (720). Typically, the wellbore plan is generated based on best available information from a geophysical model associated with the geo-physical properties of the subsurface (e.g., wave speed or velocity, density, attenuation, anisotropy), geomechanical models encapsulating stress conditions in a subterranean region of interest, the trajectory of any existing wellbores (which it may be desirable to avoid), and the existence of other drilling hazards, such as shallow gas pockets, over-pressure zones, and active fault planes. Furthermore, the wellbore plan may take into account other engineering constraints such as the maximum wellbore curvature (“dog-log”) that the drillstring may tolerate and the maximum torque and drag values that the drilling system may tolerate.

The wellbore path planning system (718) may comprise one or more computer processors in communication with computer memory containing the geophysical and geomechanical models, the reservoir simulation, information relating to drilling hazards, and the constraints imposed by the limitations of the drillstring (706) and the drilling system (700). The wellbore path planning system (718) may further include dedicated software to determine the planned wellbore path and associated drilling parameters, such as the planned wellbore diameter, the location of planned changes of the wellbore diameter, the planned depths at which casing will be inserted to support the wellbore and to prevent formation fluids entering the wellbore, and the drilling mud weights (densities) and types that may be used during drilling the wellbore. A wellbore may be drilled, guided by the wellbore path, using the drilling system (700).

While the reservoir modeler (722), reservoir simulator (732), and wellbore path planning system (718) are shown at the drilling system (700) location, in some embodiments, these elements may be remote from the drilling system (700) location. In some embodiments, the reservoir modeler (722), reservoir simulator (732), and wellbore path planning system (718) may include one or more computer systems that are similar to the computer system (902) described below with regard to FIG. 9 and the accompanying description.

A diagram of a neural network is shown in FIG. 8. At a high level, a neural network (800) may be graphically depicted as being composed of nodes (802), where here any circle represents a node, and edges (804), shown here as directed lines. The nodes (802) may be grouped to form layers (805). FIG. 8 displays four layers (808, 810, 812, 814) of nodes (802) where the nodes (802) are grouped into columns, however, the grouping need not be as shown in FIG. 8. The edges (804) connect the nodes (802). Edges (804) may connect, or not connect, to any node(s) (802) regardless of which layer (805) the node(s) (802) is in. That is, the nodes (802) may be sparsely and residually connected. A neural network (800) will have at least two layers (805), where the first layer (808) is considered the “input layer” and the last layer (814) is the “output layer”. Any intermediate layer (810, 812) is usually described as a “hidden layer”. A neural network (800) may have zero or more hidden layers (810, 812) and a neural network (800) with at least one hidden layer (810, 812) may be described as a “deep” neural network or as a “deep learning method.” In general, a neural network (800) may have more than one node (802) in the output layer (814). In this case the neural network (800) may be referred to as a “multi-target” or “multi-output” network.

Nodes (802) and edges (804) carry additional associations. Namely, every edge is associated with a numerical value. The edge numerical values, or even the edges (804) themselves, are often referred to as “weights” or “parameters”. While training a neural network (800), numerical values are assigned to each edge (804). Additionally, every node (802) is associated with a numerical variable and an activation function. Activation functions are not limited to any functional class, but traditionally follow the form:

A=ƒ(Σ_{i∈(incoming)}[(node value)ᵢ(edge value)ᵢ]),

where i is an index that spans the set of “incoming” nodes (802) and edges (804) and ƒ is a user-defined function. Incoming nodes (802) are those that, when viewed as a graph (as in FIG. 8), have directed arrows that point to the node (802) where the numerical value is being computed. Some functions for ƒ may include the linear function ƒ(x)=x, sigmoid function

$$f ( x ) = \frac{1}{1 + {e}^{- x}} ,$$

and rectified linear unit function ƒ(x)=max (0, x), however, many additional functions are commonly employed. Every node (802) in a neural network (800) may have a different associated activation function. Often, as a shorthand, activation functions are described by the function ƒ by which it is composed. That is, an activation function composed of a linear function ƒ may simply be referred to as a linear activation function without undue ambiguity.

When the neural network (800) receives an input, the input is propagated through the network according to the activation functions and incoming node (802) values and edge (804) values to compute a value for each node (802). That is, the numerical value for each node (802) may change for each received input. Occasionally, nodes (802) are assigned fixed numerical values, such as the value of 1, that are not affected by the input or altered according to edge (804) values and activation functions. Fixed nodes (802) are often referred to as “biases” or “bias nodes” (805), displayed in FIG. 8 with a dashed circle.

In some implementations, the neural network (800) may contain specialized layers (805), such as a normalization layer, or additional connection procedures, like concatenation. One skilled in the art will appreciate that these alterations do not exceed the scope of this disclosure.

As noted, the training procedure for the neural network (800) comprises assigning values to the edges (804). To begin training the edges (804) are assigned initial values. These values may be assigned randomly, assigned according to a prescribed distribution, assigned manually, or by some other assignment mechanism. Once edge (804) values have been initialized, the neural network (800) may act as a function, such that it may receive inputs and produce an output. As such, at least one input is propagated through the neural network (800) to produce an output. Generally, a training dataset is provided the neural network for training. The training dataset is composed of inputs and associated target(s), where the target(s) represent the “ground truth”, or the otherwise desired output. The neural network (800) output is compared to the associated input data target(s). The comparison of the neural network (800) output to the target(s) is typically performed by a so-called “loss function”; although other names for this comparison function such as “error function” and “cost function” are commonly employed. Many types of loss functions are available, such as the mean-squared-error function, however, the general characteristic of a loss function is that the loss function provides a numerical evaluation of the similarity between the neural network (800) output and the associated target(s).

In some embodiments, the training dataset, that includes a collection of known chromatography data in an area of interest, a reservoir fluid sample and a surface soil sample from a hydrocarbon producing well in the area of interest may be used to generate a characteristic hydrocarbon microseepage signature or a microseepage “target” used by the neural network (800). The training dataset that includes a second surface sample and a second soil gas sample from a dry well in the area of interest may be used the characteristic background signature, which may represent the background “targets”. The first and second chromatograms may be evaluated using neural networks to determine the similarity of their specific compounds, to the targets determined from the training dataset.

The loss function may also be constructed to impose additional constraints on the values assumed by the edges (804), for example, by adding a penalty term, which may be physics-based, or a regularization term. Generally, the goal of a training procedure is to alter the edge (804) values to promote similarity between the neural network (800) output and associated target(s) over the data set. Thus, the loss function is used to guide changes made to the edge (804) values, typically through a process called “backpropagation”.

While a full review of the backpropagation process exceeds the scope of this disclosure, a brief summary is provided. Backpropagation consists of computing the gradient of the loss function over the edge (804) values. The gradient indicates the direction of change in the edge (804) values that results in the greatest change to the loss function. Because the gradient is local to the current edge (804) values, the edge (804) values are typically updated by a “step” in the direction indicated by the gradient. The step size is often referred to as the “learning rate” and need not remain fixed during the training process. Additionally, the step size and direction may be informed by previously seen edge (804) values or previously computed gradients. Such methods for determining the step direction are usually referred to as “momentum” based methods.

Once the edge (804) values have been updated, or altered from their initial values, through a backpropagation step, the neural network (800) will likely produce different outputs. Thus, the procedure of propagating at least one input through the neural network (800), comparing the neural network (800) output with the associated target(s) with a loss function, computing the gradient of the loss function with respect to the edge (804) values, and updating the edge (804) values with a step guided by the gradient, is repeated until a termination criterion is reached. Common termination criteria are reaching a fixed number of edge (804) updates, otherwise known as an iteration counter; a diminishing learning rate; noting no appreciable change in the loss function between iterations; reaching a specified performance metric as evaluated on the data or a separate hold-out dataset. Once the termination criterion is satisfied, and the edge (804) values are no longer intended to be altered, the neural network (800) is said to be “trained.”

FIG. 9 depicts a block diagram of a computer system used to provide computational functionalities associated with described algorithms, methods, functions, processes, flows, and procedures as described in this disclosure, according to one or more embodiments. The illustrated computer (902) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (902) may include a computer that includes an input device, such as a keypad, keyboard, touch screen, or other device that can accept user information, and an output device that conveys information associated with the operation of the computer (902), including digital data, visual, or audio information (or a combination of information), or a GUI.

The computer (902) can serve in a role as a client, network component, a server, a database or other persistency, or any other component (or a combination of roles) of a computer system for performing the subject matter described in the instant disclosure. The illustrated computer (902) is communicably coupled with a network (930). In some implementations, one or more components of the computer (902) may be configured to operate within environments, including cloud-computing-based, local, global, or other environment (or a combination of environments).

At a high level, the computer (902) is an electronic computing device operable to receive, transmit, process, store, or manage data and information associated with the described subject matter. According to some implementations, the computer (902) may also include or be communicably coupled with an application server, e-mail server, web server, caching server, streaming data server, business intelligence (BI) server, or other server (or a combination of servers).

The computer (902) can receive requests over network (930) from a client application (for example, executing on another computer (902) and responding to the received requests by processing the said requests in an appropriate software application. In addition, requests may also be sent to the computer (902) from internal users (for example, from a command console or by other appropriate access method), external or third-parties, other automated applications, as well as any other appropriate entities, individuals, systems, or computers.

Each of the components of the computer (902) can communicate using a system bus (903). In some implementations, any or all of the components of the computer (902), both hardware or software (or a combination of hardware and software), may interface with each other or the interface (904) (or a combination of both) over the system bus (903) using an application programming interface (API) (912) or a service layer (913) (or a combination of the API (912) and service layer (913). The API (912) may include specifications for routines, data structures, and object classes. The API (912) may be either computer-language independent or dependent and refer to a complete interface, a single function, or even a set of APIs. The service layer (913) provides software services to the computer (902) or other components (whether or not illustrated) that are communicably coupled to the computer (902). The functionality of the computer (902) may be accessible for all service consumers using this service layer. Software services, such as those provided by the service layer (913), provide reusable, defined business functionalities through a defined interface. For example, the interface may be software written in JAVA, C++, or other suitable language providing data in extensible markup language (XML) format or another suitable format. While illustrated as an integrated component of the computer (902), alternative implementations may illustrate the API (912) or the service layer (913) as stand-alone components in relation to other components of the computer (902) or other components (whether or not illustrated) that are communicably coupled to the computer (902). Moreover, any or all parts of the API (912) or the service layer (913) may be implemented as child or sub-modules of another software module, enterprise application, or hardware module without departing from the scope of this disclosure.

The computer (902) includes an interface (904). Although illustrated as a single interface (904) in FIG. 9, two or more interfaces (904) may be used according to particular needs, desires, or particular implementations of the computer (902). The interface (904) is used by the computer (902) for communicating with other systems in a distributed environment that are connected to the network (930). Generally, the interface (904) includes logic encoded in software or hardware (or a combination of software and hardware) and operable to communicate with the network (930). More specifically, the interface (904) may include software supporting one or more communication protocols associated with communications such that the network (930) or interface's hardware is operable to communicate physical signals within and outside of the illustrated computer (902).

The computer (902) includes at least one computer processor (905). Although illustrated as a single computer processor (905) in FIG. 9, two or more processors may be used according to particular needs, desires, or particular implementations of the computer (902). Generally, the computer processor (905) executes instructions and manipulates data to perform the operations of the computer (902) and any algorithms, methods, functions, processes, flows, and procedures as described in the instant disclosure.

The computer (902) also includes a memory (906) that holds data for the computer (902) or other components (or a combination of both) that can be connected to the network (930). For example, memory (906) can be a database storing data consistent with this disclosure. Although illustrated as a single memory (906) in FIG. 9, two or more memories may be used according to particular needs, desires, or particular implementations of the computer (902) and the described functionality. While memory (906) is illustrated as an integral component of the computer (902), in alternative implementations, memory (906) can be external to the computer (902).

The application (907) is an algorithmic software engine providing functionality according to particular needs, desires, or particular implementations of the computer (902), particularly with respect to functionality described in this disclosure. For example, application (907) can serve as one or more components, modules, applications, etc. Further, although illustrated as a single application (907), the application (907) may be implemented as multiple applications (907) on the computer (902). In addition, although illustrated as integral to the computer (902), in alternative implementations, the application (907) can be external to the computer (902).

There may be any number of computers (902) associated with, or external to, a computer system containing computer (902), wherein each computer (902) communicates over network (930). Further, the term “client,” “user,” and other appropriate terminology may be used interchangeably as appropriate without departing from the scope of this disclosure. Moreover, this disclosure contemplates that many users may use one computer (902), or that one user may use multiple computers (902).

In some embodiments, the data processing module (312) used in the dual soil sample processing system (300) may perform hydrocarbon microseepage processing and analysis using a first computer (902) and one or more first Applications (907) while the reservoir simulation may be conducted on a second computer (902) using one or more second Applications (907).

Although only a few example embodiments have been described in detail above, those skilled in the art will readily appreciate that many modifications are possible, including dimensions, in the example embodiments without materially departing from this invention. Accordingly, all such modifications are intended to be included within the scope of this disclosure as defined in the following claims.

What is claimed:

1. A method, comprising:
collecting, using a dual soil sampling system, a dual soil sample from a borehole,
wherein the dual soil sample comprises:
a soil sample collected with an active soil sampling device, and
a soil gas sample collected with an umbrella-shaped passive soil gas sampler;
determining, using a dual soil sample processing system, an analysis of the dual soil sample; and
determining a presence of a hydrocarbon microseepage based, at least in part, on the analysis.

2. The method of claim 1, further comprising:
stabilizing the borehole using a rigid casing lubricated with a bio-based oil prior to collecting the soil sample;
generating a reservoir model, using a reservoir modeler based, at least in part, on the hydrocarbon microseepage;
performing a reservoir simulation, using a reservoir simulator, to determine a drilling target based, at least in part, on the reservoir model;
planning a wellbore path, using a wellbore path planning system, to intersect the drilling target; and
drilling a wellbore, guided by the wellbore path, using a drilling system.

3. The method of claim 1, wherein collecting the soil gas sample with the umbrella-shaped passive soil gas sampler comprises:
deploying the umbrella-shaped passive soil gas sampler at a bottom section of the borehole;
sealing the bottom section by inflating a top inflatable rubber seal of the umbrella-shaped passive soil gas sampler;
taking the soil gas sample for a sampling period; and
retrieving the umbrella-shaped passive soil gas sampler for analysis.

4. The method of claim 3, wherein the umbrella-shaped passive soil gas sampler comprises:
an outside surface covered in a gas-permeable aquaphobic membrane that selectively allows for a passage of the soil gas sample;
a gas trapping sorbent material that partially fills the umbrella-shaped passive soil gas sampler to trap the soil gas sample; and
a fluid conduit connecting the top inflatable rubber seal to a fluid pump controlled at a surface location.

5. The method of claim 1, wherein determining the analysis of the dual soil sample comprises:
performing a mineralogical analysis on the soil sample using a fluorescence (XRF) analyzer;
determining a first chromatogram of the soil sample; and
determining a second chromatogram of the soil gas sample.

6. The method of claim 5, wherein determining the first chromatogram and the second chromatogram comprises:
creating a liquid soil extract from the soil sample and extracting the liquid soil extract using a liquid extraction device;
extracting a desorbed soil gas from the soil gas sample using a thermal desorber; and
using at least one of a triple-quadruple mass spectrometer or a time-of-flight mass spectrometer:
determining the first chromatogram from the liquid soil extract, and
determining the second chromatogram from the desorbed soil gas.

7. The method of claim 1, wherein determining the presence of the hydrocarbon microseepage comprises:
using a data processing module:
generating a first discriminant function from a training dataset, using a machine learning (ML) network to define a characteristic hydrocarbon microseepage signature;
generating a second discriminant function from the training dataset, using the ML network to define a characteristic background signature; and
interpreting a first chromatogram and a second chromatogram using the first and second discriminant function to determine the presence of the hydrocarbon microseepage.

8. The method of claim 7, wherein the first and second discriminant function comprises a processed chromatogram data.

9. The method of claim 7, wherein the training dataset comprises:
a collection of known chromatography data in an area of interest;
a reservoir fluid sample;
a first calibration soil gas sample and a first calibration soil sample from near a hydrocarbon producing well in the area of interest; and
a second calibration soil sample and a second calibration soil gas sample from near a dry well in the area of interest.

10. The method of claim 7, wherein interpreting the first chromatogram and the second chromatogram further comprises, for both the first chromatogram and the second chromatogram:
removing a background signal using the second discriminant function; and
using the first discriminant function and at least one of a classification technique to identify and quantify at least one of a targeted microseepage compound.

11. The method of claim 10, wherein the classification technique is selected from a group consisting of a multivariate statistical algorithm method including, principal component analysis, hierarchical cluster analysis, canonical variants, neural net classification, and linear discriminant analysis.

12. A system, comprising:
a borehole;
a dual soil sampling system, configured to collect a dual soil sample from the borehole comprising:
an active soil sampling device configured to collect a soil sample in a bottom section of the borehole, and
an umbrella-shaped passive soil gas sampler, configured to collect a soil gas sample in the bottom section of the borehole; and
a dual soil sample processing system, comprising:
a fluorescence (XRF) analyzer, configured to perform a mineralogical analysis of the soil sample,
a liquid extraction device, configured to create and extract a liquid soil extract from the soil sample,
a thermal desorber, configured to extract a desorbed soil gas from the soil gas sample, at least one of a triple-quadruple mass spectrometer or a time-of-flight mass spectrometer, configured to determine an analysis of the dual soil sample by determining a first chromatogram from the liquid soil extract and determining a second chromatogram from the desorbed soil gas, and
a data processing module, configured to determine a presence of a hydrocarbon microseepage based, at least in part, on the analysis.

13. The system of claim 12, further comprising:
a reservoir modeler configured to produce a reservoir model based, at least in part, on the hydrocarbon microseepage;
a reservoir simulator configured to:
produce a reservoir simulation based, at least in part, on the reservoir model, and determine a drilling target based, at least in part, on the reservoir simulation;
a wellbore path planning system configured to plan a wellbore path to intersect the drilling target of a subterranean region of interest; and
a wellbore drilling system configured to drill a wellbore guided by the wellbore path.

14. The system of claim 12, wherein the borehole comprises a rigid casing lubricated with a bio-based oil configured to stabilize the borehole prior to collecting the dual soil sample.

15. The system of claim 12, wherein the umbrella-shaped passive soil gas sampler further comprises:
a top inflatable rubber seal, configured to seal the bottom section of the borehole when inflated;
an outside surface covered in a gas-permeable aquaphobic membrane that selectively allows for a passage of the soil gas sample;
a gas trapping sorbent material that partially fills the umbrella-shaped passive soil gas sampler to trap the soil gas sample; and
a fluid conduit connecting the top inflatable rubber seal to a fluid pump controlled at a surface location.

16. The system of claim 12, wherein the data processing module, when determining the presence of the hydrocarbon microseepage, is configured to:
generate a first discriminant function from a training dataset, using a machine learning (ML) network to define a characteristic hydrocarbon microseepage signature;
generate a second discriminant function from the training dataset, using the ML network to define a characteristic background signature; and
interpret the first chromatogram and the second chromatogram using the first and second discriminant function to determine the presence of the hydrocarbon microseepage.

17. The system of claim 16, wherein the first and second discriminant function comprises a processed chromatogram data.

18. The system of claim 17, wherein the training dataset comprises:
a collection of known chromatography data in an area of interest;
a reservoir fluid sample;
a first calibration soil gas sample and a first calibration soil sample from near a hydrocarbon producing well in the area of interest; and
a second calibration soil sample and a second calibration soil gas sample from near a dry well in the area of interest.

19. The system of claim 18, wherein interpreting the first chromatogram and the second chromatogram, further comprises:
for both the first chromatogram and the second chromatogram;
removing a background signal using the second discriminant function, and
using the first discriminant function and at least one of a classification technique to identify and quantify at least one of a targeted microseepage compound.

20. The system of claim 19, wherein the classification technique is selected from a group consisting of a multivariate statistical algorithm method including, principal component analysis, hierarchical cluster analysis, canonical variants, neural net classification, and linear discriminant analysis.
